(19) United States
(12) Patent Application Publication
(10) Pub. No.: US 2010/0254920 A1
(43) Pub. Date: Oct. 7, 2010

(54) Aqueous photoprotective compositions comprising hydrophilic metal oxide nanopigments and vinylpyrrolidone homopolymers

(75) Inventors: Florence L'Alloret, Paris (FR); Jean-Thierry Simonnet, Cachan (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 11/653,392

(22) Filed: Jan. 16, 2007

Related U.S. Application Data
(63) Continuation of application No. PCT/EP05/07368, filed on Jun. 20, 2005.
(60) Provisional application No. 60/600,781, filed on Aug. 12, 2004.

(30) Foreign Application Priority Data
Jul. 13, 2004 (FR) 04/07828

(51) Int. Cl. A61K 8/37; A61K 8/19; A61Q 17/04

(52) U.S. Cl. 424/60; 424/59

(57) ABSTRACT

Stable, reduced whitening photoprotective compositions contain:
a) at least one aqueous phase,
b) at least hydrophilic metal oxide nanoparticles, and
c) at least one vinylpyrrolidone homopolymer, formulated into d) a topically applicable, physiologically acceptable medium therefor.

### CROSS-REFERENCE TO PRIORITY/PCT/PROVISIONAL APPLICATIONS

This application claims priority under 35 U.S.C. §119 of FR 04/07828, filed Jul. 13, 2004, and of Provisional Application No. 60/600,781, filed Aug. 12, 2004, and is a continuation of PCT/EP 2005/007368 filed Jun. 20, 2005 and designating the United States, published in the English language as WO 2006/005521 A1 on Jan. 19, 2006, each hereby expressly incorporated by reference and each assigned to the assignee hereof.

### CROSS-REFERENCE TO COMPANION APPLICATION

Copending U.S. patent application No. [Attorney Docket No. 1016800-000698.001], filed concurrently herewith and assigned to the assignee hereof.

### BACKGROUND OF THE INVENTION

1. Technical Field of the Invention

The present application relates to photoprotective compositions comprising, formulated into a physiologically acceptable medium:

a) at least one aqueous phase,

b) at least hydrophilic inorganic nanopigments based on metal oxides untreated with an aluminum phosphate,

c) at least one vinylpyrrolidone homopolymer.

2. Description of Background and/or Related and/or Prior Art

It is well known that light radiation with wavelengths of from 280 nm to 400 nm makes possible browning of the human epidermis and that radiation with wavelengths of from 280 nm to 320 nm, known as UV-B radiation, causes skin burns and erythemas which can harm the development of natural tanning; this UV-B radiation must therefore be screened out from the skin.

It is also known that UV-A radiation, with wavelengths of from 320 nm to 400 nm, which causes browning of the skin, is capable of promoting a detrimental change in the skin, in particular in the case of sensitive skin or of skin continually exposed to solar radiation. UV-A radiation causes in particular a loss of elasticity of the skin and the appearance of wrinkles, resulting in premature aging. It promotes the triggering of the erythemal reaction or accentuates this reaction in certain individuals and can even be the cause of phototoxic or photoallergic reactions. It is therefore desirable to screen out UV-A radiation as well.

Numerous cosmetic compositions intended for the photoprotection of the skin have been provided to date.

There currently exists on the market various types of sunscreen agents: inorganic particles and organic screening agents. These screening agents must be able to absorb or block harmful solar radiation while remaining innocuous to the user.

Numerous organic sunscreen agents capable of more or less selectively absorbing harmful UV radiation have been provided to date in the field of cosmetics. However, for various reasons, these screening agents are not entirely satisfactory.

This is the reason attempts are increasingly being made to avoid the use of these organic screening agents while favoring the use of inorganic metal oxide nanoparticles, in particular nano titanium oxides and nano zinc oxides, which also act as sunscreen agents, mainly by scattering/reflecting UV radiation, while providing greater safety for the user. These nanopigments generally have a mean size for the unit particle of less than 500 nm and preferably of less than 100 nm. The metal oxide nanoparticles generally used in the formulations may be hydrophilic or else may be hydrophobic.

Certain types of metal oxide nanoparticles and more particularly those of titanium oxide and those of zinc oxide have a tendency to whiten the skin after application. This phenomenon is undesirable from an aesthetic viewpoint.

In addition to this undesirable whitening phenomenon, metal oxide nanoparticles are generally difficult to formulate in aqueous compositions and more particularly in vehicles of the emulsion type which are the most commonly used in anti-sun/sunscreen cosmetics. They have a tendency to induce a phenomenon of sedimentation, to form large aggregates and to destabilize the aqueous formulations comprising them.

Provision has already been made, in JP 2001-48731, to surface treat inorganic particles with a polyvinylpyrrolidone in order to improve their dispersibility and their stability in aqueous vehicles. However, it does not disclose metal oxide nanoparticles with a mean size for the unit particle of less than 500 nm and does not make it possible to solve the problem of whitening produced by these nanoparticles on application.

WO 2004/104111 discloses emulsions based on metal oxide micropigments treated with an aluminum phosphate in combination with an organic polymer of the polyvinylpyrrolidone type as dispersing agent.

Furthermore, the assignee hereof during its research studies, has found that polyvinylpyrrolidones do not make it possible to improve the dispersibility of hydrophobically treated metal oxide nanoparticles in the aqueous phase.

Need thus continues to exist for novel aqueous anti-sun/sunscreen formulations based on metal oxide nanoparticles for which, on the one hand, the phenomena of whitening on the skin are substantially reduced, indeed even halted, and, on the other hand, a good stability is exhibited (good dispersibility of the nanoparticles in the aqueous phase).

### SUMMARY OF THE INVENTION

It has now surprisingly and unexpectedly been determined that the above objectives could be achieved by formulating the combination of hydrophilic metal oxide nanoparticles untreated with an aluminum phosphate and of a vinylpyrrolidone hompolymer.

Thus, the present invention features photoprotective compositions comprising, formulated into a physiologically acceptable medium:

a) at least one aqueous phase,

b) at least hydrophilic metal oxide nanoparticles untreated with an aluminum phosphate, and

c) at least one vinylpyrrolidone homopolymer.

The present invention also features formulating at least one vinylpyrrolidone hompolymer into photoprotective compositions comprising at least one aqueous phase and at least hydrophilic metal oxide nanoparticles untreated with an aluminum phosphate for the purpose of reducing the whitening and/or of improving the stability of the said compositions (dispersibility of the nanoparticles in the aqueous phase).

The term “photoprotective composition” means a composition capable of screening out UV radiation, in particular solar radiation.

The term “physiologically acceptable medium” means a non-toxic medium capable of being applied to the skin, lips, hair, eyelashes, eyebrows or nails. The compositions of the invention can constitute, in particular, cosmetic or dermatological compositions.

The term “nanoparticles” means particles for which the mean size of the unit particles is less than 500 nm and preferably less than 100 nm.

The term “hydrophilic” means particles which, introduced into an aqueous phase at 25° C., at a concentration by weight of 1%, make it possible to obtain a solution which is macroscopically homogeneous to the naked eye.

### BRIEF DESCRIPTION OF THE DRAWINGS

FIG. 1 is a series of photographs illustrating the aqueous dispersions of Examples 9 to 12 to follow, and

FIG. 2 is a series of photographs illustrating the dispersions of Examples 13 to 16 to follow.

### DETAILED DESCRIPTION OF BEST MODE AND SPECIFIC/PREFERRED EMBODIMENTS OF THE INVENTION

The hydrophilic metal oxide nanoparticles used in the present invention are powders composed of particles having a mean size for the unit particle preferably of from 5 nm to 500 nm, more preferably from 10 nm to 100 nm and preferentially still from 15 nm to 50 nm.

The metal oxides forming these nanoparticles are preferably selected from titanium oxides, zinc oxides or their mixtures, whether treated or untreated.

The treated metal oxide nanoparticles are generally subjected to one or more surface treatments of a chemical, electronic, mechanochemical and/or mechanical nature with at least one compound capable of rendering them hydrophilic, such as those described, for example, in Cosmetics & Toiletries, February 1990, Vol. 105, pp. 53-64. Mention may be made, for example, of amino acids, C₁-C₅ alkanolamines, silicon oxides (silica), metal oxides, such as alumina, sodium hexametaphosphate or glycerol or their mixtures.

Mention may be made, among the treated titanium oxide nanoparticles which can be used according to the invention, of the titanium oxide nanoparticles treated with at least one coating agent, such as:

silica and alumina, such as the products “Microtitanium Dioxide MT 500 SA”, and “Microtitanium Dioxide MT 100 SA” from Tayca and the products “Tioveil Fin”, “Tioveil OP”, “Tioveil MOTG” and “Tioveil IPM” from Tioxide, the product “Mirasun TIW 60” from Rhodia, the product “Sunveil PW-6030A-20” from CClC or the product “Solaveil CT-10W” from Uniquema;

sodium hexametaphosphate, such as the product “Microtitanium dioxide MT 150W” from Tayca,

alumina and glycerol, such as the product “UVT-M212” from Kemira,

alumina, silica and alginic acid, such as “Microtitanium dioxide MT 100 AQ” from Tayca.

The untreated titanium oxide nanopigments are, for example, those marketed by Tayca under the trademarks “Microtitanium dioxide MT 500 B” or “Microtitanium dioxide MT600 B” from Degussa under the trademark “P 25”, by Wacker under the trademark “Oxyde de titane transparent PW” by Miyoshi Kasei under the trademark “UFTR”, by Tomen under the trademark “ITS” and by Tioxide under the trademark “Tioveil AQ”.

The untreated zinc oxide nanoparticles are for example:

those marketed under the trademark “Z-cote” by Sunsmart;

those marketed under the trademark “Nanox” by Elementis;

those marketed under the trademark “Nanoguard WCD 2025” by Nanophase Technologies.

Also exemplary are coated mixtures of titanium dioxide and of zinc dioxide, such as that coated with alumina, with silica and with glycerol, for example the product “M 211” marketed by Kemira.

Very particularly preferred are titanium oxide nanoparticles, whether amorphous or in crystalline (rutile and/or anatase) form and whether treated or untreated.

The hydrophilic metal oxide nanoparticles in accordance with the invention are preferably present in the compositions according to the invention in proportions ranging from 0.5% to 30% by weight, with respect to the total weight of the composition, and preferably ranging from 1% to 25% by weight, with respect to the total weight of the composition.

Exemplary vinylpyrrolidone homopolymers according to the invention include the following polymers:

| | Number-average | |
|---|---|---|
| Commercial name | molar mass (g/mol) | Supplier |
| Luviskol K 17 Powder | 2500 | BASF |
| Kollidon 17 PF | 2500 | BASF |
| Kollidon 12 PF | 1300 | BASF |
| Kollidon 30 | 10,000 | BASF |
| Polyvinylpyrrolidone K 60 | 160,000 | Fluka |
| Solution | | |
| Kollidon 90 | 360,000 | BASF |

According to a particularly preferred embodiment of the invention, the vinylpyrrolidone hompolymers have a molar mass of less than 20,000 g/mol and better still of less than 10,000 g/mol.

The concentration by weight of vinylpyrrolidone homopolymer in the composition preferably varies from 0.01% to 10%, more preferably from 0.1% to 5% to better still from 0.2% to 2.5%.

The ratio by weight of the metal oxide nanoparticles to the polyvinylpyrrolidone preferably varies from 1 to 30, more preferably from 5 to 20 and particularly from 8 to 12.

The metal oxide nanoparticles can be brought into contact with a polyvinylpyrrolidone directly during the formulation of the composition, for example by considering an aqueous phase comprising the particles and the polymer introduced at the end of formulation. The metal oxide nanoparticles can also be pretreated with the polyvinylpyrrolidone before the introduction into a composition; this pretreatment can be carried out by dispersing the particles in an aqueous polyvinylpyrrolidone solution, followed by evaporation of the water, in order to recover a powder of nanoparticles coated with a polymer.

According to a preferred embodiment of the invention, the ionic strength of the aqueous phase of the composition, without the metal oxide, is less than 0.1 mol/l. The ionic strength is determined using a CDM 230 conductivity meter (Meterlab) resulting in the conductivity of the medium, a parameter related to the ionic strength by the following relationship:

Ionic strength (mol/l)=0.1×Conductivity (Siemens/cm)+0.19

Furthermore, the compositions in accordance with the invention can comprise other additional organic photoprotective agents active in the UV-A and/or UV-B region which are water-soluble or fat-soluble or else insoluble in the cosmetic solvents commonly employed.

The additional organic photoprotective agents are selected in particular from among cinnamic derivatives; anthranilates; salicylic derivatives; dibenzoylmethane derivatives; camphor derivatives; benzophenone derivatives; β,β-diphenylacrylate derivatives; triazine derivatives; benzotriazole derivatives; benzalmalonate derivatives; benzimidazole derivatives; imidazolines; bis-benzoazolyl derivatives, as disclosed in EP-669,323 and U.S. Pat. No. 2,463,264; p-aminobenzoic acid (PABA) derivatives; methylenebis(hydroxyphenylbenzotriazole) derivatives, as disclosed in U.S. Pat. Nos. 5,237,071, 5,166,355, GB-2,303,549, DE-197,26,184 and EP-893,119; benzoxazole derivatives, as disclosed in EP-0,832,642, EP-1,027,883, EP-1,300,137 and DE-10162844; screening polymers and screening silicones, such as those disclosed in particular in WO 93/04665; dimers derived from a-alkylstyrene, such as those disclosed in DE 19855649; 4,4-diarylbutadienes, as disclosed in EP-0,967,200, DE-19746654, DE-19755649, EP-A-1,008,586, EP-1,133,980 and EP-133,981; and mixtures thereof.

Examples of such additional organic photoprotective agents are those denoted below under their INCl names:

Cinnamic Derivatives:

Ethylhexyl Methoxycinnamate, marketed in particular under the trademark “Parsol MCX” by Hoffmann-LaRoche,

### Isopropyl Methoxycinnamate,

Isoamyl Methoxycinnamate, marketed under the trademark “Neo Heliopan E 1000” by Haarmann and Reimer,

### Cinoxate,

### DEA Methoxycinnamate,

### Diisopropyl Methyl Cinnamate,

### Glyceryl Ethylhexanoate Dimethoxycinnamate.

Dibenzoylmethane Derivatives:

Butyl Methoxydibenzoylmethane, marketed in particular under the trademark “Parsol 1789” by Hoffmann-LaRoche,

### Isopropyl Dibenzoylmethane.

pAra-Aminobenzoic Acid Derivatives:

### PABA,

### Ethyl PABA,

### Ethyl Dihydroxypropyl PABA,

Ethylhexyl Dimethyl PABA, marketed in particular under the trademark “Escalol 507” by ISP,

### Glyceryl PABA,

PEG-25 PABA, marketed under the trademark “Uvinul P25” by BASF.

Salicylic Derivatives:

Homosalate, marketed under the trademark “Eusolex HMS” by Rona/EM Industries,
Ethylhexyl Salicylate, marketed under the trademark “Neo Heliopan OS” by Haarmann and Reimer,
Dipropyleneglycol Salicylate, marketed under the trademark “Dipsal” by Scher,
TEA Salicylate, marketed under the trademark “Neo Heliopan TS” by Haarmann and Reimer.

β,β-Diphenylacrylate Derivatives:

Octocrylene, marketed in particular under the trademark “Uvinul N539” by BASF,
Etocrylene, marketed in particular under the trademark “Uvinul N35” by BASF.

Benzophenone Derivatives:

Benzophenone-1, marketed under the trademark “Uvinul 400” by BASF,
Benzophenone-2, marketed under the trademark “Uvinul D50” by BASF,
Benzophenone-3 or Oxybenzone, marketed under the trademark “Uvinul M40” by BASF,
Benzophenone-4, marketed under the trademark “Uvinul MS40” by BASF,

### Benzophenone-5,

Benzophenone-6, marketed under the trademark “Helisorb 11” by Norquay,
Benzophenone-8, marketed under the trademark “Spectra-Sorb UV-24” by American Cyanamid,
Benzophenone-9, marketed under the trademark “Uvinul DS-49” by BASF,

### Benzophenone-12,

n-Hexyl 2-(4-diethylamino-2-hydroxybenzoyl)benzoate.

Benzylidenecamphor Derivatives:

3-Benzylidene camphor, manufactured under the trademark “Mexoryl SD” by Chimex,
4-Methylbenzylidene camphor, marketed under the trademark “Eusolex 6300” by Merck,
Benzylidene Camphor Sulfonic Acid, manufactured under the trademark “Mexoryl SL” by Chimex,
Camphor Benzalkonium Methosulfate, manufactured under the trademark “Mexoryl SO” by Chimex,
Terephthalylidene Dicamphor Sulfonic Acid, manufactured under the trademark “Mexoryl SX” by Chimex,
Polyacrylamidomethyl Benzylidene Camphor, manufactured under the trademark “Mexoryl SW” by Chimex.

Phenylbenzimidazole Derivatives:

Phenylbenzimidazole Sulfonic Acid, marketed in particular under the trademark “Eusolex 232” by Merck,
Disodium Phenyl Dibenzimidazole Tetrasulfonate, marketed under the trademark “Neo Heliopan AP” by Haarmann and Reimer.

Phenylbenzotriazole Derivatives:

Drometrizole Trisiloxane, marketed under the trademark “Silatrizole” by Rhodia Chimie,
Methylene Bis-Benzotriazolyl Tetramethylbutylphenol, marketed in the solid form under the trademark “Mixxim BB/100” by Fairmount Chemical or in the micronized form in aqueous dispersion under the trademark “Tinosorb M” by Ciba Specialty Chemicals.

Triazine Derivatives:

Bis-Ethylhexyloxyphenol Methoxyphenyl Triazine, marketed under the trademark “Tinosorb S” by Ciba-Geigy,
Ethylhexyl triazone, marketed in particular under the trademark “Uvinul T150” by BASF,
Diethylhexyl Butamido Triazone, marketed under the trademark “Uvasorb HEB” by Sigma 3V,
2,4,6-Tris(dineopentyl 4′-aminobenzalmalonate)-s-triazine,
2,4,6-Tris(diisobutyl 4′-aminobenzalmalonate)-s-triazine.

Anthranilic Derivatives:

Menthyl anthranilate, marketed under the trademark “Neo Heliopan MA” by Haarmann and Reimer.

Imidazoline Derivatives:

### Ethylhexyl Dimethoxybenzylidene Dioxoimidazoline Propionate.

Benzalmalonate Derivatives:

Polyorganosiloxanes with benzalmalonate functional groups, such as Polysilicone-15, marketed under the trademark “Parsol SLX” by Hoffmann-LaRoche.

4,4-Diarylbutadiene Derivatives:
1,1-Dicarboxy(2,2′-dimethylpropyl)-4,4-diphenylbutadiene.

Benzoxazole Derivatives:
2,4-Bis[5-1(dimethylpropyl)benzoxazol-2-yl-(4-phenyl)-imino]-6-(2-ethylhexyl)imino-1,3,5-triazine, marketed under the trademark Uvasorb K2A by Sigma 3V;
   and mixtures thereof.

The preferred additional organic photoprotective agents are selected from among:
Ethylhexyl Methoxycinnamate,
Ethylhexyl Salicylate,
Homosalate,
Octocrylene,
Phenylbenzimidazole Sulfonic Acid,
Benzophenone-3,
Benzophenone-4,
Benzophenone-5,
n-Hexyl 2-(4-d iethylamino-2-hydroxybenzoyl)benzoate,
4-Methylbenzylidene camphor,
Terephthalylidene Dicamphor Sulfonic Acid,
Disodium Phenyl Dibenzimidazole Tetrasulfonate,
Methylene Bis-benzotriazolyl Tetramethylbutylphenol,
Bis-Ethylhexyloxyphenol Methoxyphenyl Triazine,
Ethylhexyl triazone,
Diethylhexyl Butamido Triazone,
2,4,6-Tris(dineopentyl 4′-aminobenzalmalonate)-s-triazine,
2,4,6-Tris(diisobutyl 4′-aminobenzalmalonate)-s-triazine,
Drometrizole Trisiloxane,

Polysilicone-15,
1,1-Dicarboxy(2,2′-dimethylpropyl)-4,4-diphenylbutadiene,
2,4-Bis[5-1(dimethylpropyl)benzoxazol-2-yl-(4-phenyl)-imino]-6-(2-ethylhexyl)imino-1,3,5-triazine,
   and mixtures thereof.

The aqueous compositions of the invention can be provided in all the forms generally used for a topical application, in particular in the form of an oil-in-water emulsion (direct emulsion), water-in-oil emulsion (inverse emulsion), water-in-oil-in-water emulsion (multiple emulsion) or also of an aqueous gel.

The compositions of the invention can comprise all the additives commonly used in cosmetics and will find applications in the care and makeup field and in the field of anti-sun/sunscreen products.

The additional photoprotective agents are generally present in the compositions according to the invention in proportions ranging from 0.01% to 20% by weight, with respect to the total weight of the composition, and preferably ranging from 0.1% to 10% by weight, with respect to the total weight of the composition.

The aqueous compositions in accordance with the present invention can additionally comprise conventional cosmetic adjuvants selected in particular from among fatty substances, organic solvents, ionic or non-ionic and hydrophilic or lipophilic thickening agents, softening agents, humectants, opacifiers, stabilizers, emollients, silicones, anti-foaming agents, fragrances, preservatives, anionic, cationic, non-ionic, zwitterionic or amphoteric surfactants, active principles, fillers, polymers, propellants, basifying or acidifying agents or any other ingredient commonly employed in the cosmetics and dermatological field.

The fatty substances can be an oil or a wax or their mixtures. The term “oil” means a compound which is liquid at ambient temperature. The term “wax” means a compound which is solid or substantially solid at ambient temperature and which has a melting point generally of greater than 35° C.

Exemplary oils are mineral oils (liquid paraffin); vegetable oils (sweet almond oil, macadamia oil, blackcurrant seed oil or jojoba oil); synthetic oils, such as perhydrosqualene, fatty alcohols, fatty amides (such as isopropyl lauroyl sarcosinate, marketed under the trademark of “Eldew SL-205” by Ajinomoto), fatty acids or esters (such as the benzoate of C₁₂-C₁₅ alcohols marketed under the trademark “Finsolv TN” or “Witconol TN” by Witco, octyl palmitate, isopropyl lanolate, triglycerides, including those of capric/carprylic acids, or the dicaprylyl carbonates marketed under the trademark “Cetiol CC” by Cognis), or oxyethylenated or oxypropylenated fatty esters and ethers; silicone oils (cyclomethicone, polydimethylsiloxanes or PDMSs) or fluorinated oils; or polyalkylenes.

Exemplary waxy compounds are paraffin wax, carnauba wax, beeswax or hydrogenated castor oil.

Exemplary organic solvents are the lower alcohols and polyols.

The latter can be selected from glycols and glycol ethers, such as ethylene glycol, propylene glycol, butylene glycol, dipropylene glycol or diethylene glycol.

Exemplary hydrophilic thickening agents are carboxyvinyl polymers, such as Carbopols (carbomers) and Pemulens (acrylate/C₁₀-C₃₀ alkyl acrylate copolymer); polyacrylamides, such as, for example, the crosslinked copolymers marketed under the trademarks Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) by Seppic; polymers and copolymers of 2-acrylamido-2-methylpropanesulfonic acid, optionally crosslinked and/or neutralized, such as the poly(2-acrylamido-2-methylpropanesulfonic acid) marketed by Hoechst under the trademark “Hostacerin AMPS” (CTFA name: ammonium polyacryldimethyltauramide); cellulose derivatives, such as hydroxyethylcellulose; polysaccharides and in particular gums, such as xanthan gum; and mixtures thereof.

Exemplary lipophilic thickening agents are synthetic polymers, such as the poly(C₁₀₋₃₀ alkyl acrylate) marketed under the trademark “Doresco IPA 13-1” by Landec, or also modified clays, such as hectorite and its derivatives, for example the products marketed under the Bentone names.

And exemplary active principles include:

vitamins (A, C, E, K, PP, and the like) and their derivatives or precursors, alone or as mixtures,

agents for combating pollution and/or free radicals;

depigmenting agents and/or propigmenting agents;

anti-glycation agents;

soothing agents;

NO-synthase inhibitors;

agents which stimulate the synthesis of dermal or epidermal macromolecules and/or which prevent their decomposition;

agents which stimulate the proliferation of fibroblasts;

agents which stimulate the proliferation of keratinocytes;

muscle relaxants;

tightening agents;

mattifying agents;

keratolytic agents;

desquamating agents;

moisturizing agents;

anti-inflammatories;

agents which act on the energy metabolism of the cells;

insect repellents;

substance P or CRGP antagonists;

agents for combating hair loss and/or for promoting hair regrowth;

anti-wrinkle agents.

Of course, one skilled in this art will take care to choose the optional additional compound or compounds mentioned above and/or their amounts such that the advantageous properties intrinsically attached to the compositions in accordance with the invention are not, or not substantially, detrimentally affected by the envisaged addition or additions.

The compositions according to the invention can be prepared according to techniques well known to one skilled in this art. They can in particular be provided in the form of a simple or complex emulsion (O/W, W/O, O/W/O or W/O/W), such as a cream, a milk or a cream gel; in the form of an aqueous gel; or in the form of a lotion. They can optionally be packaged in an aerosol and be provided in the form of a foam or spray.

Preferably, the compositions according to the invention are provided in the form of an oil-in-water or water-in-oil emulsion.

The emulsions generally comprise at least one emulsifier selected from among amphoteric, anionic, cationic or non-ionic emulsifiers, used alone or as a mixture. The emulsifiers are appropriately selected according to the emulsion to be obtained (W/O or O/W).

Exemplary emulsifying surfactants which can be used for the preparation of the W/O emulsions include sorbitan alkyl esters or ethers, glycerol alkyl esters or ethers or sugar alkyl esters or ethers; or silicone surfactants, such as dimethicone copolyols, for example the mixture of cyclomethicone and of dimethicone copolyol marketed under the trademark “DC 5225 C” by Dow Corning, and alkyl dimethicone copolyols, for example lauryl methicone copolyol, marketed under the trademark “Dow Corning 5200 Formulation Aid” by Dow Corning, cetyl dimethicone copolyol, for example the product marketed under the trademark Abil EM 90® by Goldschmidt, and the mixture of cetyl dimethicone copolyol, of polyglyceryl (4 mol) isostearate and of hexyl laurate marketed under the trademark Abil WE 09 by Goldschmidt. One or more coemulsifiers which can advantageously be selected from the group consisting of polyol alkyl esters can also be added thereto.

Exemplary polyol alkyl esters include polyethylene glycol esters, such as PEG-30 dipolyhydroxystearate, for example the product marketed under the trademark Arlacel P135 by ICI.

Exemplary glycerol and/or sorbitan esters include polyglyceryl isostearate, such as the product marketed under the trademark Isolan GI 34 by Goldschmidt; sorbitan isostearate, such as the product marketed under the trademark Arlacel 987 by ICI; the isostearate of sorbitan and of glycerol, such as the product marketed under the trademark Arlacel 986 by ICI, and mixtures thereof.

For the O/W emulsions, exemplary emulsifiers include non-ionic emulsifiers, such as oxyalkylenated (more particularly polyoxyethylenated) esters of fatty acids and of glycerol; oxyalkylenated esters of fatty acids and of sorbitan; oxyalkylenated (oxyethylenated and/or oxypropylenated) esters of fatty acids, such as the PEG-100 stearate/glyceryl stearate mixture marketed, for example, by ICI under the trademark Arlacel 165; oxyalkylenated (oxyethylenated and/or oxypropylenated) ethers of fatty alcohols; sugar esters, such as sucrose stearate; fatty alcohol and sugar ethers, in particular alkyl polyglucosides (APG), such as decyl glucoside and lauryl glucoside, marketed, for example, by Henkel under the respective names Plantaren 2000 and Plantaren 1200, cetearyl glucoside, optionally as a mixture with cetearyl alcohol, marketed, for example, under the trademark Montanov 68 by Seppic, under the trademark Tegocare CG90 by Goldschmidt and under the trademark Emulgade KE3302 by Henkel, and arachidyl glucoside, for example in the form of the mixture of arachidyl and behenyl alcohols and of arachidyl glucoside marketed under the trademark Montanov 202 by Seppic. According to a specific embodiment of the invention, the mixture of the alkyl polyglucoside as defined above with the corresponding fatty alcohol can be in the form of a self-emulsifying composition, as disclosed, for example, in WO-A-92/06778.

When an emulsion is involved, the aqueous phase thereof can comprise a non-ionic vesicular dispersion prepared according to known processes (Bangham, Standish and Watkins, J. Mol. Biol., 13, 238 (1965), FR-2,315,991 and FR-2,416,008).

The compositions according to the invention are applied in a large number of treatments, whether regime or regimen, in particular cosmetic treatments, to the skin, lips and/or hair, including the scalp, in particular for the protection and/or the care of the skin, lips and/or hair and/or for making up the skin and/or lips.

The present invention also features formulating the compositions as defined above for the manufacture of products for the cosmetic treatment of the skin, lips, nails, hair, eyelashes, eyebrows and/or scalp, in particular care products, sun protection products and makeup products.

The cosmetic compositions according to the invention can, for example, be administered as care and/or sun protection products for the face and/or body with a liquid to semi-liquid consistency, such as lotions, milks, relatively smooth creams, gels or cream gels. They can optionally be packaged in an aerosol and be provided in the form of a foam or spray.

The cosmetic compositions according to the invention can, for example, be used as makeup products.

The compositions according to the invention in the form of vaporizable fluid lotions are applied to the skin or the hair in the form of fine particles using pressurization devices. The devices in accordance with the invention are well known to this art and comprise non-aerosol pumps or “atomizers”, aerosol containers comprising a propellant and aerosol pumps using compressed air as propellant. The latter are disclosed in U.S. Pat. Nos. 4,077,441 and 4,850,517.

The compositions packaged in an aerosol in accordance with the invention generally comprise conventional propellants, such as, for example, hydrofluorinated compounds, dichlorodifluoromethane, difluoroethane, dimethyl ether, isobutane, n-butane, propane or trichlorofluoromethane. They are preferably present in amounts ranging from 15 to 50% by weight, with respect to the total weight of the composition.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Examples 1 to 4

### Aqueous Dispersions Comprising 20% as AM of Hydrophilic Titanium Oxide Nanoparticles (Mirasun TiW 60) in the Presence of 2.2% of Polymer, at pH 5:

The polymers used are as follows:

A: Polyacrylic acid, 2,000 g/mol (outside the invention)

B: Anhydride and diisobutylene copolymer in the sodium salt form supplied under the trademark of “Orotan 731 DP” by Rohm & Haas (outside the invention)

C: Polyvinylpyrrolidone with a mass of 2,500 g/mol, supplied under the trademark of “Luviskol K17 Powder” from BASF (invention)

1) Dispersion Examples 1, 2, 3 and 4 are Prepared According to the Following Protocol:

2.2 g of polymer are dissolved with stirring for 1 hour in 37 grams of deionized water, the pH of which is adjusted to 5 with the appropriate amount of citric acid. 9 grams of water are introduced into 50 grams of a 40% aqueous dispersion of hydrophilic TiO₂ nanoparticles (Mirasun TiW 60) and the pH is adjusted to 5 with the appropriate amount of citric acid. The two preceding aqueous phases are mixed by simple stirring for 30 minutes.

2) The Stability of the Aqueous Dispersions Obtained is Evaluated by Measuring their Viscosity According to the Following Test Procedure:

The viscosity measurements are carried out 24 hours after the preparation of the dispersions using a Haake RS150 rheometer equipped with cone/plate geometry (35 mm, 2°) and with a thermostatically-controlled bath in order to control the temperature. The measurements are carried out in the flow mode, the stress being varied from 0.001 and 1 Pa according to 15 stationary phases of 120 s distributed logarithmically. The value of the viscosity for a shear rate of 10 s⁻¹ is then measured. The lower the viscosity, the better the state of dispersion of the nanoparticles in the aqueous phase.

| | | Viscosity (Pa · s) at a shear |
|---|---|---|
| Examples | Polymer | rate of 10 s⁻¹ |
| 1 (control) | None | 0.01 |
| 2 (outside the | A | 0.03 |
| invention) | | |
| 3 (outside the | B | 0.11 |
| invention) | | |
| 4 (invention) | C | 0.004 |

Only the use of the polyvinylpyrrolidone C according to the invention made it possible to improve the stability of the aqueous dispersion.

### Examples 5 and 6

### Aqueous Dispersions Comprising 10% as AM (Active Material) of Titanium Oxide (Mirasun TiW 60) in the Presence of Salt and at pH 5

The dispersions are prepared according to the same procedure as for Example 1 and the rheological measurements are also carried out in the same way. The state of dispersion is also evaluated by microscopic observation.

| | Ionic strength | Viscosity (Pa · s) at a |
|---|---|---|
| Examples | (mol/l) | shear rate of 10 s⁻¹ |
| 5 (invention) | 0 | 0.002 |
| 6 (outside the | 0.17 | 0.015 |
| invention) | | |

The aqueous dispersion 6, the ionic strength of which is equal to 0.17 mol/l, is less stable than the aqueous dispersion 5, the ionic strength of which is zero.

### Examples 7 and 8

### Sun Milks

| | Example 7 | |
|---|---|---|
| | (outside the | Example 8 |
| Ingredients | invention) | (invention) |
| Octocrylene | 8 | 8 |
| Butylmethoxydibenzoylmethane | 2 | 2 |
| C₁₂₋₁₅ Alkyl benzoate | 4 | 4 |
| Cyclohexadimethylsiloxane | 10 | 10 |
| Ethanol | 4 | 4 |
| Diglycol/CHDM/Isophthalates/SIP | 2 | 2 |
| copolymer | | |
| Glycerol | 2 | 2 |
| Triethanolamine | 0.3 | 0.3 |
| Titanium oxide, “Mirasun TiW 60”, | 4 (AM) | 4 (AM) |
| from Rhodia | | |
| Polyvinylpyrrolidone with a mass of | 0 | 0.44 |
| 2500 g/mol, supplied under the | | |
| trademark of “Luviskol K17 Powder” | | |
| from BASF | | |
| Citric acid, pH 6.9 | 0.1 | 0.1 |
| Deionized water | q.s. for 100 | q.s. for 100 |

1) Method of Preparation of the Two Sun Milks:

The emulsion is prepared by rapid introduction of the aqueous phase into the oily phase with stirring using a homogenizer of Moritz type at a stirring speed of 3,000 rpm over 15 minutes. The emulsion is then cooled to ambient temperature and then subjected to strong shearing using a homogenizer of Rannie type under a pressure of 600 bar. A white and fluid milk is obtained.

For Example 7, 10 grams of Mirasun TiW 60, the pH of which is adjusted beforehand to 7 with the appropriate amount of citric acid, are introduced into the emulsion.

For Example 8, the polyvinylpyrrolidone is dissolved, with stirring for 1 hour, in 20 grams of deionized water; 10 grams of Mirasun TiW 60, the pH of which is adjusted beforehand to 7 with the appropriate amount of citric acid, are introduced into this solution with simple stirring over 30 minutes. This solution is then introduced with stirring into the emulsion.

2) Measurement of the Whitening on the Skin:

30 mg of each of the formulations are applied to an area of 4 cm×3 cm of the inner face of the forearm and are spread using the finger with 10 rotational movements.

The area of the skin treated with each of the formulations is observed with the naked eye after application.

The whitening of the skin is also measured using a spectrocolorimeter (CM 2002 from Minolta) which makes it possible to determine the brightness B of the deposit. The reduction in the whitening is evaluated by calculating the following ratio R, expressed as percentages:

R=100×(B_{TiO}_{₂}−B_{TiO}_{₂}_{+polymer})/(B_{TiO}_{₂}−B_{bare skin})

According to this test, the reduction in the whitening is regarded as significant if it is at least equal to 10%.

It is observed with the naked eye that the composition 8 according to the invention, comprising hydrophilic TiO₂ nanoparticles in the presence of polyvinylpyrrolidone, produces a substantially lower whitening in comparison with the composition 7, which does not comprise polyvinylpyrrolidone.

This phenomenon was confirmed by the measurements of qthe brightness and the calculation of the reduction in the whitening.

| Compositions | R (%) |
|---|---|
| Control: | / |
| Bare skin | |
| Example 7 | / |
| (outside the invention) | |
| Example 8 | 45.7 |
| (invention) | |

### Examples 9 to 16

### Aqueous Dispersions Comprising 10% as AM of Titanium Oxide Nanoparticles Treated or not Treated at the Surface with a Polyvinylpyrrolidone

The TiO₂ nanoparticles used are:

“Mirasun TiW 60”, supplied by Rhodia as hydrophilic titanium oxide nanoparticles (alumina and silica coating);

“UV Titan M 262”, supplied by Kemira as hydrophobic titanium oxide nanoparticles (PDMS coating).

The polyvinylpyrrolidones used are:

“Luviskol K17 Powder” (number-average molar mass 2,500 g/mol),

“Kollidon 12 PF” (number-average molar mass 1,300 g/mol), supplied by BASF,

“Polyvinylpyrrolidone K 60 solution” (number-average molar mass 160,000 g/mol), supplied by Fluka.

The following eight examples of aqueous dispersions of TiO₂ nanoparticles were prepared:

| Compositions | TiO₂ used | PVP used |
|---|---|---|
| Example 9 | Mirasun TiW 60 | None |
| (outside the invention) | (hydrophilic) | |
| Example 10 | Mirasun TiW 60 | Luviskol K17 Powder |
| (invention) | (hydrophilic) | MW: 2,500 |
| Example 11 | Mirasun TiW 60 | Kollidon 12 PF |
| (invention) | (hydrophilic) | MW: 1,300 |
| Example 12 | Mirasun TiW 60 | Polyvinylpyrrolidone |
| (invention) | (hydrophilic) | K 60 Solution |
| | | MW: 160,000 |
| Example 13 | UV Titan M 262 | None |
| (outside the invention) | (hydrophobic) | |
| Example 14 | UV Titan M 262 | Luviskol K17 Powder |
| (outside the invention) | (hydrophobic) | MW: 2,500 |
| Example 15 | UV Titan M 262 | Kollidon 12 PF |
| (outside the invention) | (hydrophobic) | MW: 1,300 |
| Example 16 | UV Titan M 262 | Polyvinylpyrrolidone |
| (outside the invention) | (hydrophobic) | K 60 Solution |
| | | MW: 160,000 |

1) Method of Preparation of the TiO₂ Powders Treated with a Polyvinylpyrrolidone:

100 ml of a 1% aqueous polyvinylpyrrolidone (PVP) solution are prepared by dissolution of 1 gram of PVP in 99 grams of deionized water at 25° C. for 2 hours with mechanical stirring. 100 ml of a 10% as AM suspension of nano titanium oxide are prepared by dilution with water, the pH being adjusted to 7 using the appropriate amount of citric acid. The preceding 200 ml are mixed and milled for 48 hours using a bead mill. The aqueous phase is then evaporated under reduced pressure until a fine white powder is obtained.

2) Method of Preparation of the Aqueous Dispersion Examples Nos. 9 to 16:

10 grams of powder are dispersed in 90 grams of a 1% aqueous Carbopol 980 (Noveon) gel at pH 7; the dispersion is produced using an Ultra-Turrax mixer equipped with the 25 F rod for 10 minutes at a speed of 24,000 rpm.

3) Microscopic Characterization of these Aqueous Dispersions:

For each of Examples 9 to 16, the state of dispersion of the titanium oxide nanoparticles was evaluated by microscopic observation.

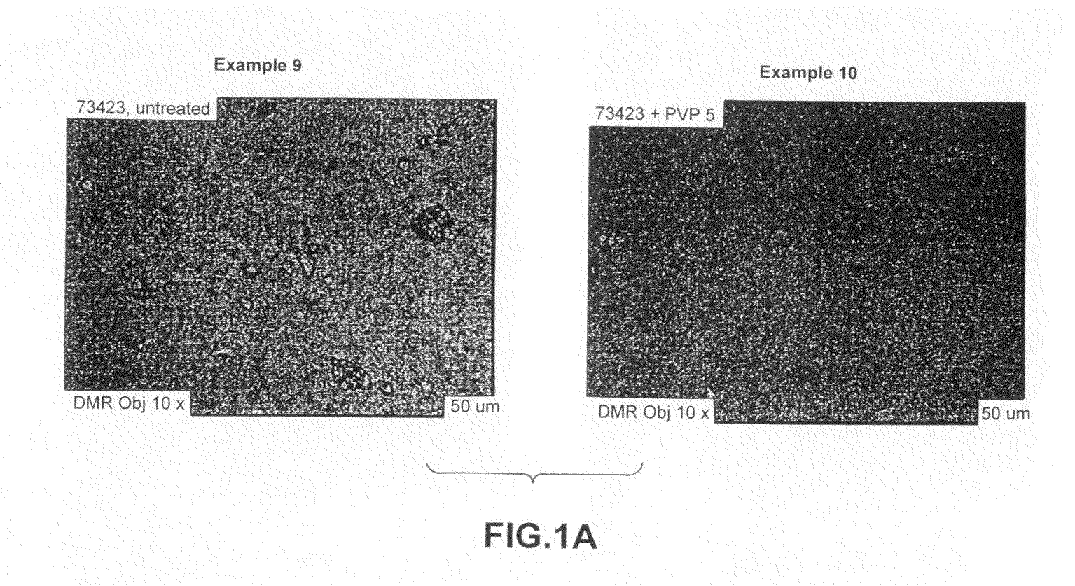

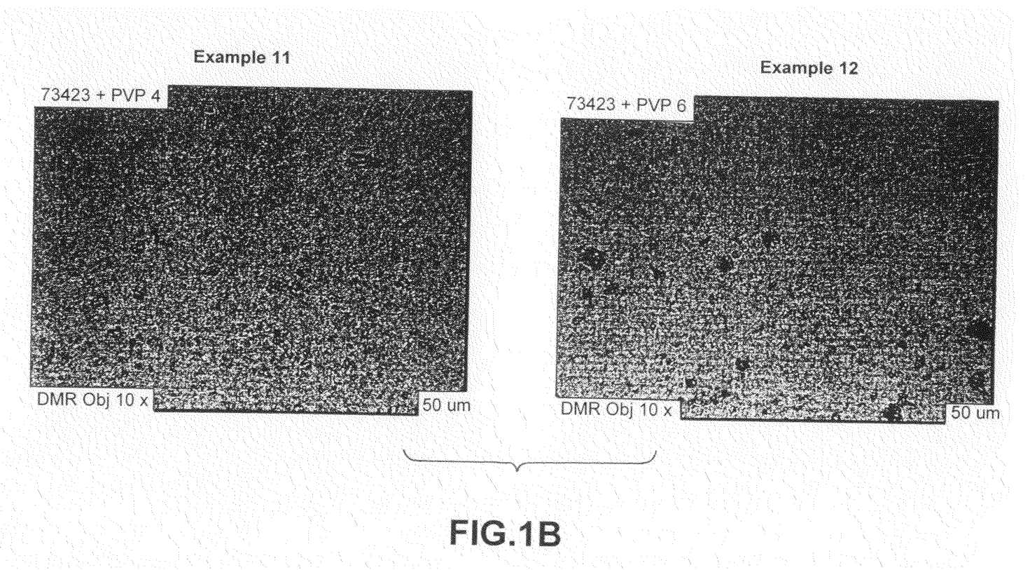

The photographs of the aqueous dispersion Examples 9 to 12 observed are represented in FIG. 1.

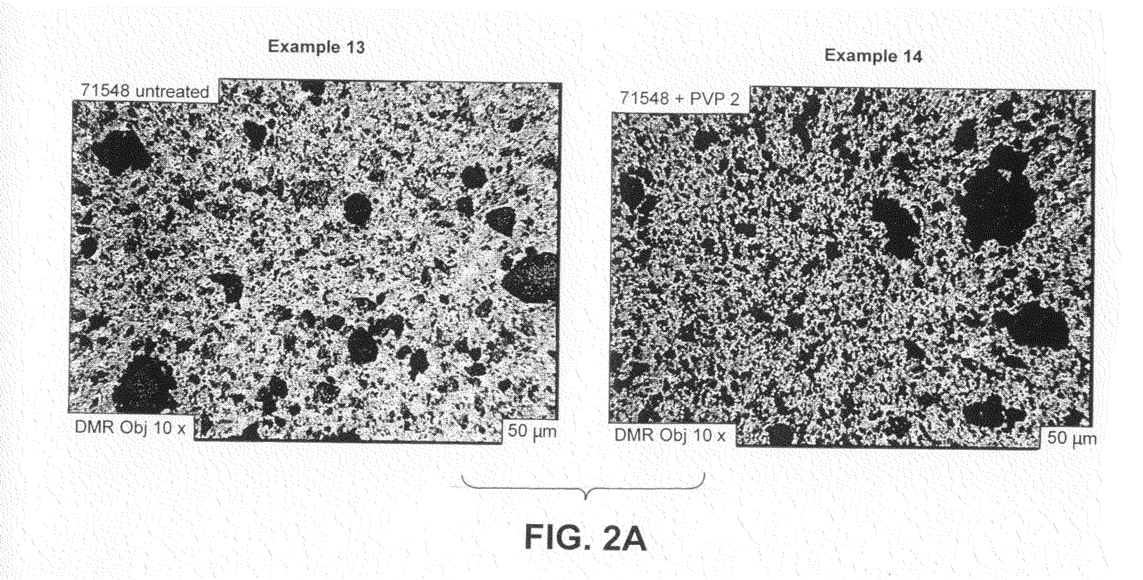

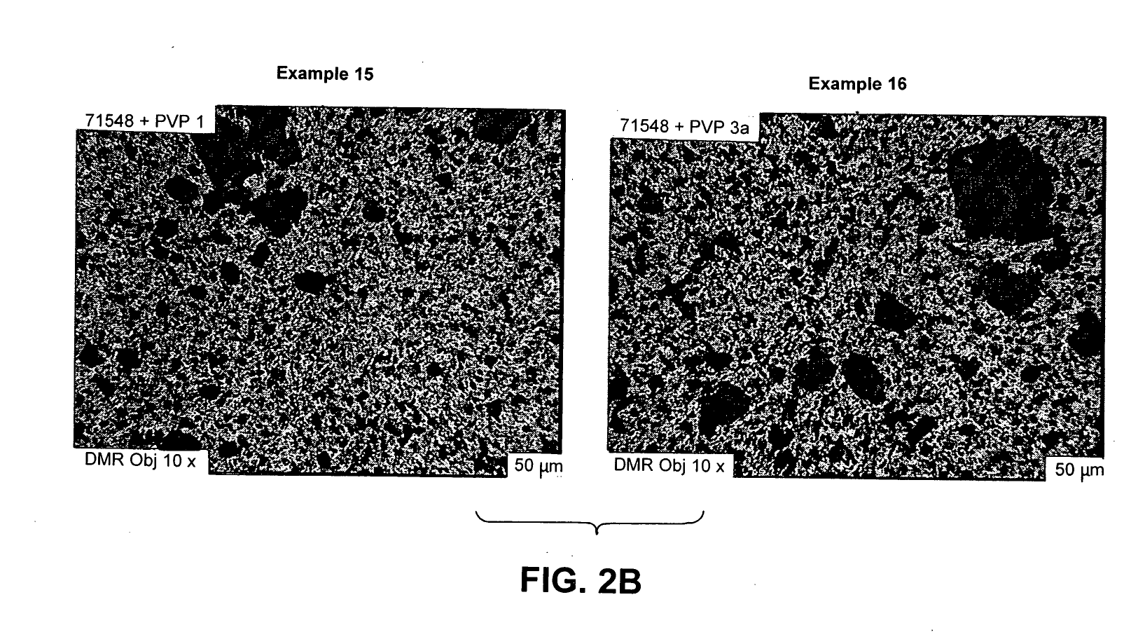

The photographs of the dispersion Examples 13 to 16 observed are represented in FIG. 2.

In FIG. 1, it is found that, generally, the introduction of a polyvinylpyrrolidone improves the state of dispersion of the hydrophilic titanium oxide nanoparticles (Examples 10, 11 and 12, in comparison with the control Example 9 not comprising PVP).

In contrast, it is found, in FIG. 2, that the addition of the polyvinylpyrrolidone has no influence on the state of dispersion of the hydrophobic titanium oxide nanoparticles (Examples 14, 15 and 16 with the control Example 13 not comprising PVP).

Furthermore, the photographs of Examples 10, 11 and 12 according to the invention show that the state of dispersion of the hydrophilic TiO₂ nanoparticles with the addition of the polymers “Luviskol K17 Powder” and “Kollidon 12 PF” having a molecular mass of less than 20,000 g/mol is better than that obtained with the polymer “Polyvinylpyrrolidone K 60 Solution” with a molecular mass of 160,000 g/mol.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable photoprotective composition, comprising:
a) at least one aqueous phase,
b) at least hydrophilic inorganic nanopigments based on metal oxides untreated with an aluminum phosphate, and
c) at least one vinylpyrrolidone homopolymer, formulated into d) a topically applicable, physiologically acceptable medium therefor.

2. The photoprotective composition as defined by claim 1, said hydrophilic metal oxide nanoparticles having a mean size for the unit particle of less than 500 nm.

3. The photoprotective composition as defined by claim 1, said hydrophilic metal oxide nanoparticles having a mean size for the unit particle of from 5 nm to 500 nm.

4. The photoprotective composition as defined by claim 1, the metal oxides forming said nanoparticles comprising titanium oxides, zinc oxides or mixtures thereof, whether treated or untreated.

5. The photoprotective composition as defined by claim 1, said hydrophilic metal oxide nanoparticles comprising nanoparticles treated with at least one coating agent that renders same hydrophilic.

6. The photoprotective composition as defined by claim 5, said at least one coating agent comprising amino acids, C₁-C₅ alkanolamines, silicon oxides, metal oxides, sodium hexametaphosphate, glycerol or mixtures thereof.

7. The photoprotective composition as defined by claim 1, said hydrophilic metal oxide nanoparticles comprising titanium oxide nanoparticles, whether amorphous or in crystalline (rutile and/or anatase) form and whether treated or untreated.

8. The photoprotective composition as defined by claim 1, the hydrophilic metal oxide nanoparticles comprising from 0.5% to 30% by weight thereof.

9. The photoprotective composition as defined by claim 1, said at least one vinylpyrrolidone homopolymer having a molar mass of less than 20,000 g/mol.

10. The photoprotective composition as defined by claim 1, wherein the concentration of vinylpyrrolidone homopolymer ranges from 0.01% to 10% by weight thereof.

11. The photoprotective composition as defined by claim 1, the ratio by weight of the metal oxide nanoparticles to the at least one polyvinylpyrrolidone polymer ranging from 1 to 30.

12. The photoprotective composition as defined by claim 1, wherein said metal oxide nanoparticles are pretreated with the at least one vinylpyrrolidone hompolymer prior to formulation therein.

13. The photoprotective composition as defined by claim 1, wherein the ionic strength of the aqueous phase thereof, without the metal oxide, is less than 0.1 mol/l.

14. The photoprotective composition as defined by claim 1, further comprising at least one additional organic photoprotective agent active in the UV-A and/or UV-B region which is water-soluble or fat-soluble or insoluble in the common cosmetic solvents.

15. The photoprotective composition as defined by claim 14, said at least one additional organic photoprotective agent being selected from the group consisting of cinnamic derivatives; anthranilates; salicylic derivatives; dibenzoylmethane derivatives; camphor derivatives; benzophenone derivatives; β,β-diphenylacrylate derivatives; triazine derivatives; benzotriazole derivatives; benzalmalonate derivatives; benzimidazole derivatives; imidazolines; bis-benzoazolyl derivatives; p-aminobenzoic acid (PABA) derivatives; methylenebis(hydroxyphenylbenzotriazole) derivatives; benzoxazole derivatives; screening polymers and screening silicones; dimers derived from a-alkylstyrene; 4,4-diarylbutadienes; and mixtures thereof.

16. The photoprotective composition as defined by claim 15, said at least one additional organic photoprotective agent being selected from the group consisting of:
Ethylhexyl Methoxycinnamate,
Ethylhexyl Salicylate,
Homosalate,
Octocrylene,
Phenylbenzimidazole Sulfonic Acid,
Benzophenone-3,
Benzophenone-4,
Benzophenone-5,
n-Hexyl 2-(4-diethylamino-2-hydroxybenzoyl)benzoate,
4-Methylbenzylidene camphor,
Terephthalylidene Dicamphor Sulfonic Acid,
Disodium Phenyl Dibenzimidazole Tetrasulfonate,
Methylene Bis-benzotriazolyl Tetramethylbutylphenol,
Bis-Ethylhexyloxyphenol Methoxyphenyl Triazine,
Ethylhexyl triazone,
Diethylhexyl Butamido Triazone,
2,4,6-Tris(dineopentyl 4′-aminobenzalmalonate)-s-triazine,
2,4,6-Tris(diisobutyl 4′-aminobenzalmalonate)-s-triazine,
Drometrizole Trisiloxane,
Polysilicone-15,
1,1-Dicarboxy(2,2′-dimethylpropyl)-4,4-diphenylbutadiene,
2,4-Bis[5-1(dimethylpropyl)benzoxazol-2-yl-(4-phenyl)-imino]-6-(2-ethylhexyl)imino-1,3,5-triazine,
and mixtures thereof.

17. The photoprotective composition as defined by claim 14, said at least one additional organic photoprotective agent comprising from 0.01% to 20% by weight thereof.

18. The photoprotective composition as defined by claim 1, further comprising at least one cosmetic adjuvant selected from the group consisting of fatty substances, organic solvents, ionic or non-ionic and hydrophilic or lipophilic thickening agents, softening agents, humectants, opacifiers, stabilizers, emollients, silicones, anti-foaming agents, fragrances, preservatives, anionic, cationic, non-ionic, zwitterionic or amphoteric surfactants, active principles, fillers, polymers, propellants, basifying or acidifying agents, and mixtures thereof.

19. The photoprotective composition as defined by claim 1, formulated as a simple or complex emulsion; an aqueous gel; or a lotion.

20. The photoprotective composition as defined by claim 19, formulated as an oil-in-water or water-in-oil emulsion.

21. A regime or regimen for photoprotecting the skin, lips, nails, hair, eyelashes, eyebrows and/or scalp against the damaging effects of UV radiation, comprising topically applying thereon a thus effective amount of the photoprotective composition as defined by claim 1.

22. The photoprotective composition as defined by claim 1, formulated as a care and/or anti-sun protection product.

23. The photoprotective composition as defined by claim 1, formulated as a makeup product.

24. The method comprising formulating at least one vinylpyrrolidone homopolymer as defined in claim 1 into such photoprotective composition comprising at least one aqueous phase and at least hydrophilic metal oxide nanoparticles untreated with an aluminum phosphate for the purpose of reducing the whitening and/or of improving the stability of the said composition.

25. The photoprotective composition as defined by claim 2, said hydrophilic meal oxide nanoparticles having a mean size for the unit particle of less than 100 nm.
